(19) United States
(12) Patent Application Publication
(10) Pub. No.: US 2023/0200937 A1
(43) Pub. Date: Jun. 29, 2023

(54) HYBRID CERAMIC PROSTHESIS

(71) Applicant: HASS CO., LTD., Gangneung-si (KR)

(72) Inventors: Hwan Soon KOH, Anyang-si (KR); Kyung Sik OH, Incheon (KR); Young Pyo HONG, Gangneung-si (KR); Duk Won HAM, Gangneung-si (KR); Yong Su KIM, Gangneung-si (KR)

(73) Assignee: HASS CO., LTD., Gangneung-si (KR)

(21) Appl. No.: 18/089,120

(22) Filed: Dec. 27, 2022

(30) Foreign Application Priority Data
Dec. 28, 2021 (KR) 10-2021-0190081
Dec. 2, 2022 (KR) 10-2022-0166704
Dec. 2, 2022 (KR) 10-2022-0166705

(51) Int. Cl. A61C 5/77; A61K 6/802; A61C 13/265; A61L 27/10; A61L 27/06; A61L 27/14

(52) U.S. Cl. CPC A61C 5/77; A61K 6/802; A61C 13/2656; A61L 27/10; A61L 27/06; A61L 27/14; A61F 2002/30065

(57) ABSTRACT

Proposed is a hybrid ceramic prosthesis. Since the prosthesis is made of a novel hybrid ceramic material, the prothesis has a similar color and transmittance to natural teeth, minimizes tooth removal during preparation for placement of the prothesis, is securely attached to the target tooth by snap-fit fastening to prevent detachment of the prothesis and re-treatment caused by the prothesis detachment, and can be more conveniently applied to the patient than conventional prostheses, enabling an one-day-one-stop dental procedure, thereby reducing burden to patients. The prothesis is useful as a primary crown.

### CROSS REFERENCE TO RELATED APPLICATION

The present application claims priority to Korean Patent Application No. 10-2021-0190081, filed Dec. 28, 2021, Korean Patent Application No. 10-2022-0166704, filed Dec. 2, 2022, Korean Patent Application No. 10-2022-0166705, filed Dec. 2, 2022 the entire contents of which is incorporated herein for all purposes by this reference.

### BACKGROUND OF THE disclosure

### 1. Field of the Disclosure

The present disclosure relates to a hybrid ceramic prosthesis and provides a rapidly implantable hybrid ceramic prosthesis that has aesthetically similar color and transmittance to natural teeth and enables a one-day-one-stop dental procedure by minimizing tooth removal, thereby reducing the burden of dental treatment.

### 2. Description of the Related Art

In humans, the blue-white primary teeth grow from around 6 to 8 months after birth. Generally, the primary teeth occur in the order of central incisor, lateral incisor, first primary molar, canine, and second primary molar. Finally, a total of 20 primary teeth come out by the age of 2-3 years. From around the age of 7, the primary teeth naturally fall out in a first-come first-out fashion and are sequentially replaced by permanent teeth.

However, since the primary teeth have a larger contact surface and more space between teeth than permanent teeth, the primary teeth are very prone to cavities. When permanent teeth grow, the existing primary teeth serve as a guide. Therefore, in the case of having a tooth decay, partial removal and replacement with a prosthesis are carried out rather than tooth extraction. In severe cases, a space maintainer is inserted after tooth extraction to secure space for the permanent teeth to come out. An artificial crown that restores a damaged primary tooth is called a primary crown, and ready-made crowns having various sizes are mainly used.

Primary crowns made of various materials are used, but the most commonly used products are stainless steel crowns (SSC). Since stainless steel is chemically stable, there is little risk of corrosion or discoloration. Moreover, since stainless steel is inexpensive, SSC products are the most preferred products. However, the SSC products are limitedly used only in the posterior teeth because SSC products are aesthetically disadvantageous in terms of color difference with the surrounding teeth.

As existing crowns, there are resin-veneered SSC products having the structure in which a composite resin is overlaid on metal, and composite strip crown products having the structure in which resin cement is charged into a transparent polymer tube of a tooth model. However, these products have limitations in aesthetic or functional aspects.

Recently, white dental crowns made of Zirconia, which have a color similar to natural teeth and have high strength, have been used to compensate for the aesthetic disadvantage of the other existing products. However, these crowns have lower transmittance and thus look more opaque than the other existing products when illuminated. In addition, due to the high strength, when the crown is applied to a tooth cavity, a large amount of tooth removal is required, which may impose a burden to young patients.

U.S. Pat. No. 8,651,867 discloses a dental crown that is made of a thermoplastic polymer material, can be rapidly installed, can be easily removed or replaced, can be mass-produced, and can also be used for toddlers.

### LITERATURE OF RELATED ART

### Patent Literature

(Patent Literature 1) U.S. Pat. No. 8,651,867 (2014 Feb., 18)

### SUMMARY OF THE DISCLOSURE

An objective of the present disclosure is to provide a rapidly implantable hybrid ceramic prosthesis that has similar color and transmittance to natural teeth and enables a one-day-one-stop dental prosthesis procedure by m inimizing tooth removal during dental treatment.

The present disclosure provides a hybrid ceramic prosthesis including a thermoplastic polymer matrix and ceramic particles dispersed in the matrix.

In a hybrid ceramic prosthesis according to one embodiment of the present disclosure, the thermoplastic polymer matrix may include one or more high performance thermoplastics selected from the group consisting of polyether ether ketone (PEEK), polyphenylsulfone (PPSU), polyamide-imide (PAI), polyphenylene sulfide (PPS), polysulfone (PSU), and polyether sulfone (PES). In a hybrid ceramic prosthesis according to one preferred embodiment of the present disclosure, the thermoplastic polymer matrix may include polyphenylsulfone (PPSU).

In a hybrid ceramic prosthesis according to one preferred embodiment of the present disclosure, the polyphenylsulfone (PPSU) exhibits a light transmittance of at least 75%, a haze of not higher than 5.1%, and a yellowness index of 28 or lower when a film thickness thereof is 2 mm or less. In addition, the polyphenylsulfone (PPSU) exhibits a tensile strength of 68 to 72 MPa, a tensile modulus of 2.0 to 2.4 GPa, a tensile elongation at break of 60% to 120%, a flexural strength of 88 to 95 MPa, and an impact resistance (Izod impact, notched) of 680 to 700 J/m when the film thickness thereof is 2 mm or less.

In a hybrid ceramic prosthesis according to one preferred embodiment of the present disclosure, the ceramic particles may be included in an amount of 3 to 50 parts by weight with respect to 100 parts by weight of the thermoplastic polymer matrix.

In a hybrid ceramic prosthesis according to one preferred embodiment of the present disclosure, the ceramic particles may be silicate-based glass ceramic particles. Specifically, the silicate-based glass ceramic particles may be lithium disilicate glass ceramic particles or barium silicate glass ceramic particles.

The hybrid ceramic prosthesis according to one embodiment of the present disclosure may further include a white pigment. Specifically, the white pigment may include at least one selected from the group consisting of titanium dioxide (TiO₂), zinc oxide (ZnO), and zinc phosphate (Zn₃(PO₄)₂). In a preferred embodiment, the white pigment may include titanium dioxide (TiO₂).

In a hybrid ceramic prosthesis according to one preferred embodiment, the white pigment may be included in an amount of 0.05 to 1.0 part by weight with respect to 100 parts by weight of the thermoplastic polymer matrix.

The hybrid ceramic prosthesis according to one preferred embodiment may be an extrusion molded product including 3.0 to 50.0 parts by weight of barium silicate and 0.05 to 1.0 part by weight of titanium dioxide (TiO₂) , with respect to 100 parts by weight of the polyphenylsulfone (PPSU).

According to one preferred embodiment of the present disclosure, the prosthesis may be a primary crown. More particularly, the prosthesis may be a prosthesis that can be fastened to a prepared tooth by snap-fit fastening.

The hybrid ceramic prosthesis of the present disclosure has the following advantages: being aesthetically similar color and transmittance to natural teeth; having a moderate strength, thereby minimize the amount of tooth erosion in dental treatment;

having strength to withstand deformation of a material and elasticity to recover from the deformation whereby the prosthesis can be firmly fastened to a target teeth by snap-fit fastening, thereby reducing the burden of re-treatment attributable to prosthesis detachment; being more conveniently implanted than existing counterparts and enabling a one-day-one-stop dental procedure, thereby reducing burden to young patients who may have fear of a dental treatment environment.

### BRIEF DESCRIPTION OF THE DRAWINGS

FIGS. 1A to 1E are photographs of the appearance of samples made of PPSU polymer alone, sample made of a mixture of a polymer and ceramic particles, and sample made of a mixture of a polymer, ceramic particles, and a white pigment;

FIGS. 2A and 2B are photographs of a snap-fit-on state in which a hybrid ceramic prosthesis obtained by injection molding of pellets having a shape illustrated in FIG. 1E is combined with a tooth model, and FIG. 2C is a photograph of a snap-fit-off state in which a ceramic prosthesis is separated from a tooth model;

FIG. 3 is a graph illustrating the measurement results of hardness and wear resistance of a hybrid ceramic prosthesis produced by injection molding; and

FIG. 4 is a schematic diagram of an apparatus for measuring wear resistance.

### DESCRIPTION OF THE PREFERRED EMBODIMENTS

Hereinafter, embodiments and examples will be described in detail such that those skilled in the art can easily understand and reproduce the present disclosure. The embodiments and examples described below are provided only to aid those skilled in the art in fully understanding the present disclosure, and thus can be modified in various other forms, and the scope of the present disclosure is not limited thereto.

As used herein, the singular forms “a”, “an”, and “the” may include plural forms, unless the context clearly dictates otherwise. In addition, as used herein, “comprise” and/or “comprising” specify the presence of shapes, numbers, steps, operations, members, elements, and/or combinations thereof, and do not preclude the presence or addition of one or more other shapes, numbers, operations, elements, and/or combinations thereof.

Ceramics have been used as dental restorative materials for a long time because of their excellent biocompatibility, good chemical durability, color stability, and similar transmittance to natural teeth. However, there is a problem that ceramic blocks can easily break because they have greater flexural strength and less elasticity than natural teeth.

In other words, since the occlusion in the oral cavity is influenced by vertical loads as well as lateral pressure, it is necessary to repair the broken prosthesis according to various kinetic situations, personal anomaly, or diet, etc. Although the recent advent of the CAD/CAM system has reduced the time required for clinical practice, it is still a burden to patients that the patients visit the dentist several times for re-treatment, especially when the patients are children who may have more fear of dental treatment than adults.

Therefore, it is an objective of the present disclosure to provide a prosthesis that can be manufactured in a ready-made rather than a customized form and thus can be rapidly applied on site in a clinical procedure without additional machining.

In this respect, the hybrid ceramic prosthesis according to the disclosure may be a primary crown.

In the present disclosure, the hybrid ceramic prosthesis and a target tooth can be firmly fastened by a snap-fit physical connection and a chemical bond whereby the burden of re-treatment caused by a prosthesis detachment can be reduced. With the snap-fit fastening, the prosthesis of the present disclosure can be more conveniently applied than conventional counterparts because the application of the prosthesis is achieved simply by fitting the prosthesis to the teeth in clinical practice.

A snap-fit is an assembling method in which two components are easily fastened without using additional components or fasteners. In the description above and below, the term “snap-fit” is defined as a fastening method in which a prepared tooth, which is cut to be ready for placement of a restorative material, and a prosthesis are physically fastened through the sequential steps of deformation, fixation, and restoration of the prosthesis without using any components or fasteners.

When fitting a prosthesis by the snap-fit fastening, by simply undercutting a side portion of the prosthesis, the prepared tooth and the prosthesis can be easily fastened due to the strength and elastic properties of the prosthesis.

In order for a prosthesis to be fastened to a tooth by the snap-fit fastening, the prosthesis is required to be made of a material having strength to endure the stress during the deformation phase and elasticity to recover after the deformation phase. Generally, a material with higher strength has lower elasticity. When a material has high strength and lower elasticity, the material breaks when stress higher than a critical level is applied thereto. Therefore, a material that is appropriate in both physical properties, i.e. strength and elasticity, is required.

To meet the requirement, i.e., balancing between strength and elasticity, the present disclosure provides a hybrid ceramic prosthesis including a thermoplastic polymer matrix and ceramic particles dispersed in the matrix. Conventionally, a photocurable urethane acrylate has been used as a dental repair or prosthetic material. This material has the advantage of being photocurable, which can speed the treatment or prosthesis preparation and satisfy the physical properties of a tooth substitute. However, when a snap-fit prosthesis is made of a urethane acrylate polymer, the prosthesis has good strength but poor elastic properties and poor deformation response. It is therefore preferred that in the present disclosure, the matrix is made of a thermoplastic polymer and specifically a high performance thermoplastics. The high performance thermoplastics are high-performance plastics that can replace metals. The high performance thermoplastics highly heat-resistant to be permanently used in a temperature environment of 150° C. to 300° C. or higher, and are good in strength and elasticity. In addition, the high performance thermoplastics have other good properties such as weight reduction and chemical resistance.

In the hybrid ceramic prostheses according to the present disclosure, the thermoplastic polymer matrix may comprise such high performance thermoplastics and more particularly one or materials selected from the group consisting of polyether ether ketone (PEEK), polyphenylsulfone (PPSU), polyamide-imide (PAI), polyphenylene sulfide (PPS), polysulfone (PSU), and polyether sulfone (PES).

Among polyether ether ketones (PEEK), medical PEEKs are high-performance materials that maintain excellent mechanical properties at temperatures above 100° C. and is highly resistant to common sterilization techniques. In other words, for sterilization with more than 1,500 cycles, the mechanical properties thereof are not impaired, and phenomena such as bleaching, discoloration (yellowing), and calcification are not observed.

In addition, such plastics exhibit the best safety in medical applications as well as excellent chemical resistance, so that they are widely applied to surgical instruments in joint restoration, traumatology, and orthopedics, and also have wide applications in dental markets and other fields.

Therefore, when PEEK is included in a polymer matrix of a material for a hybrid ceramic prosthesis, the material can maintain the physical properties thereof even when undergoing high-temperature heat treatment during the manufacturing of a prosthesis, has good chemical resistance and biocompatibility, is not bleached or discolored. Therefore, a dental prosthesis that is aesthetically good can be obtained from the material.

In addition, PPSU is an amorphous plastic having a high glass transition temperature and low water absorption. The PPSU has a high modulus of elasticity as well as has higher impact resistant and chemical resistant than polysulfone (PSU) or polyether sulfone (PES). In addition, it is widely applied in the medical field because it has excellent resistance to high-temperature steam sterilization and to detergents and disinfectants compared to other product groups, and it is also widely used as a baby bottle material that requires high heat resistance and high durability.

Table 1 below shows that PPSU can be sterilized with more kinds of sterilization methods than other polysulfone products.

TABLE 1

| | Steam | | | | | |
|---|---|---|---|---|---|---|
| | (134° C. or lower for 18 | | | Ethylene | Hydrogen | |
| | minutes) | | | oxide | peroxide | Gamma |
| | 10 | 100 | 1,000 | 100 | 200 | radiation |
| | cycles | cycles | cycles | cycles | cycles | 40 kGy |
| PPSU | V | V | V | V | V | V |
| PES | V | X | X | V | V | V |
| PSU | V | V | X | V | V | V |

* V marking means being sterilizable

In addition, PPSU is confirmed as a stable substance in tests for cytotoxicity, hypersensitivity, percutaneous toxicity, acute systemic toxicity, etc., and is a material proven to be biocompatible in many application fields as it has been used for surgical instruments, tool handles, sterilization trays, and implant trails including femoral and tibial components for knee, hip, and shoulder replacement processes.

That is, PPSU may be most preferably used as the material of the polymer matrix of the hybrid ceramic prosthesis of the present disclosure because PPSU has a high strength and elastic modulus and exhibits good biocompatibility and high heat resistance.

In particular, in the hybrid ceramic prosthesis of the present disclosure, given the aesthetic properties of the prosthesis, since the PPSU polymer constituting the polymer matrix exhibits a light transmittance of at least 75%, a haze of 5.1% or less, and a yellowness index of 28 or less when the film thickness thereof is 2 mm or less. In addition, given the mechanical properties and the function of a snap-fit prosthesis, it is preferable that the PPSU polymer satisfies the conditions:

a tensile strength of 68 to 72 MPa, a tensile modulus of 2.0 to 2.4 GPa, a tensile elongation at break of 60% to 120%, a flexural strength of 88 to 95 MPa, and an impact resistance (Izod impact, notched) of 680 to 700 J/m when the film thickness thereof is 2 mm or less.

According to the present disclosure, when a hybrid ceramic prosthesis is prepared using PEEK or PPSU as a base polymer, since the elasticity and strength are balanced, a biocompatible dental prosthesis suitable for snap-fit fastening can be obtained. In addition, the hybrid ceramic prosthesis of the present disclosure may include PAI, PPS, PSU, or PES, etc., as a polymer matrix, and such materials may be included solely or in combination of two or more.

Alternatively, the hybrid ceramic prosthesis according to the present disclosure may further include ceramic particles dispersed in the polymer matrix. The ceramic particles preferably include silicate-based glass ceramic particles. Specifically, since barium silicate glass ceramic particles or lithium silicate glass ceramic particles exhibit suitable physical properties required for as a dental prosthetic material, the barium or lithium silicate glass ceramic particles can be applied to a permanent tooth prosthesis. Specifically, lithium disilicate glass ceramic particles have good light transmittance and are aesthetic materials. Therefore, the lithium disilicate glass ceramic particles can be applied directly to the anterior teeth without having to process the surface of the teeth with a special material. In addition, the flexural strength is 400 MPa or more, the lithium disilicate glass ceramic particles can resist the bite force of the posterior teeth. In addition, since the particles can be corroded by HF (acid) and treated with silane, the particles have a strong bonding force with respect to a polymer, and the abrasion of the antagonist teeth is reduced.

As such, the hybrid ceramic prosthesis including the thermoplastic polymer matrix and the ceramic particles has adequate transmittance so that the hybrid ceramic prosthesis is not more opaque than conventional prostheses when illuminated, and has a color similar to natural teeth.

The hybrid ceramic prosthesis including the thermoplastic polymer matrix and ceramic particles dispersed in the matrix, according to the present disclosure, may have varying mechanical properties or aesthetics depending on the type of thermoplastic polymer matrix and/or the type of ceramic particles as described above, and the content of the ceramic particles dispersed in the thermoplastic polymer matrix may have an important influence on exhibiting the optimum properties to achieve snap-fit fastening. The ceramic particles are contained preferably in an amount of less than 50 parts by weight, more preferably in an amount of 3 to 50 parts by weight, and still more preferably 10.0 to 40.0 parts by weight, per 100 parts by weight of the thermoplastic polymer matrix.

The hybrid ceramic prosthesis according to the present disclosure has both the advantages of a ceramic, including excellent wear resistance, color reproducibility, and color stability, and the advantages of a polymer, including strong bonding with a resin cement and excellent processability.

In the case of placing existing ceramic prostheses having excessively high strength, since a large amount of tooth removal is required to prepare a target tooth, it takes a long time for the tooth preparation. Therefore, it is difficult to apply the conventional ceramic prostheses to young patients due to the long tooth preparation time. In the case of the hybrid ceramic prosthesis according to the present disclosure, since the hybrid ceramic prosthesis has the optimum strength and thus the tooth removal is minimized, the prosthesis can be quickly placed by snap-fit fastening, thereby reducing burden to patients.

On the other hand, the hybrid ceramic prosthesis according to the present disclosure may further include a white pigment to improve aesthetics.

Preferably, the white pigment is contained in an amount of 0.05 to 1.0 part by weight per 100 parts by weight of the polymer matrix. For example, when the polymer matrix of the hybrid ceramic prosthesis is made of PPSU, since the PPSU material is yellow, it is preferable to add a white pigment to make a color similar to a natural tooth.

Examples of the white pigment include titanium dioxide (TiO₂), zinc oxide (ZnO), and zinc phosphate (Zn₃(PO₄)₂), but are not limited thereto.

Titanium dioxide (TiO₂) is a representative white pigment and is an inorganic compound having a variety of applications such as plastics, rubber, paints, etc. used in daily lives Among white pigments, titanium dioxide has the highest refractive index and has an accurate particle size and dispersibility. In addition, titanium dioxide is a material having good obliterating power and good tinting strength. It is also a material that is highly chemically and physically stable and is non-toxic and harmless to the environment and human body.

Zinc oxide (ZnO) is a white pigment used as a sunscreen because it has a high blocking power against ultraviolet rays.

Zinc oxide (ZnO) is added to materials such as rubber and glass to increase resistance to heat because it has high heat capacity and high thermal conductivity. It is also added to ceramics to make products resistant to heat and impact. Zinc phosphate (Zn₃(PO₄)₂) is a white pigment that is applicable to enamels as well as water-soluble emulsion paints due to the good storage stability thereof. In particular, zinc phosphate has been widely used as a dental cement. In this case, zinc phosphate is mainly used for cementation of inlays, crowns and other oral devices. In addition, zinc phosphate can be used as a permanent adhesive due to the long lifespan thereof. In addition, zinc phosphate has a thermal conductivity similar to that of dentin, so it is widely used in clinical practice.

Aside from the examples, other pigments can be used, and the pigment for use in the present disclosure is not limited to the examples mentioned above.

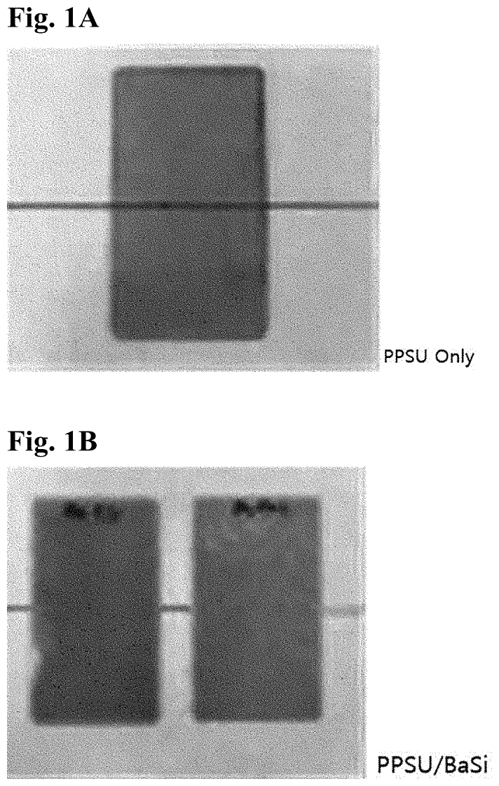

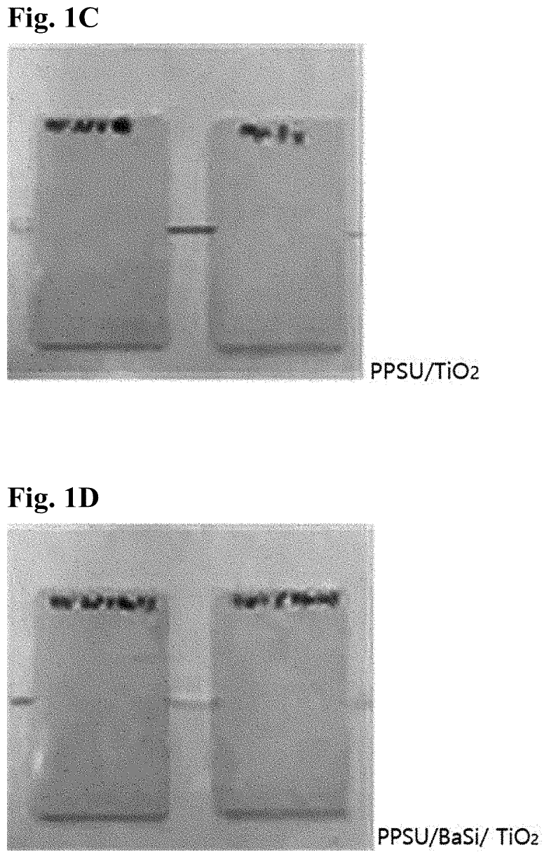

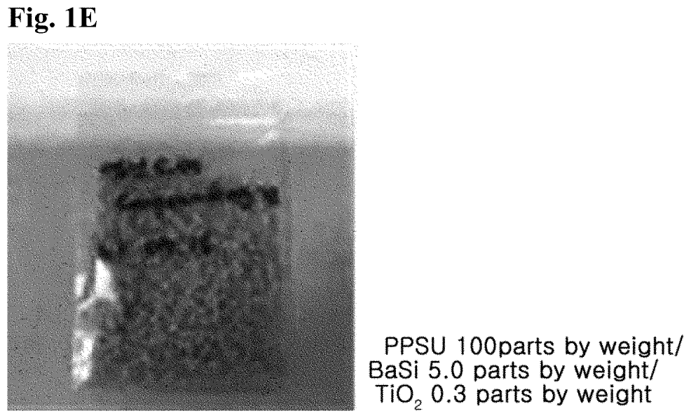

To aid understanding of the present disclosure, samples containing a thermoplastic matrix alone, samples containing a thermoplastic polymer matrix and ceramic particles dispersed in the thermoplastic polymer matrix, and samples containing a thermoplastic polymer matrix, ceramic particles dispersed in the thermoplastic polymer matrix, and a white pigment were manufactured. The color and transparency of the samples were observed with the eyes. The results are shown in FIGS. 1A to 1E.

The samples were manufactured by compounding and extruding, taking into account the thermal properties of PPSU, for each material.

FIG. 1A is a photograph of samples made of only PPSU, which is a polymer. Referring to FIG. 1A, it is seen that the samples are yellow which is the original color of PPSU.

FIG. 1B is a photograph of samples made of a mixture including PPSU and barium silicate but not including a white pigment. The left sample was made of a mixture including 5 parts by weight of barium silicate per 100 parts by weight of PPSU, and the right sample was made of a mixture including 10 parts by weight of barium silicate per 10.0 parts by weight of PPSU. As the content of barium silicate increased, the color became paler. However, but it was difficult to tell that the color was close to the natural color of the teeth.

FIG. 1C is a photograph of samples made of a mixture including PPSU and titanium dioxide (TiO₂) which is as a white pigment but not including ceramic particles. The left sample was made of a mixture including 0.3 parts by weight of titanium dioxide (TiO₂) per 100 parts by weight of PPSU, and the right sample was made of a mixture including 0.5 parts by weight of titanium dioxide (TiO₂) per 100 parts by weight of PPSU.

As the pigment content increased, the color of the sample became little closer to white. However, since the materials of the samples did not contain ceramic particles, the surface texture and the transparency of the samples were quite different from natural teeth.

FIG. 1D is a photograph of samples made of a mixture including PPSU, barium silicate, and titanium dioxide. The left sample was made of a mixture including 5.0 parts by weight of barium silicate and 0.3 parts by weight of titanium dioxide (TiO₂) per 100 parts by weight of PPSU, and the right sample was made of a mixture including 10.0 parts by weight of barium silicate and 0.3 parts by weight of titanium dioxide (TiO₂) per 100 parts by weight of PPSU.

The left and right samples exhibit somewhat different colors for each other. However, natural teeth exhibit various colors, all of the samples are found to be suitable tooth application materials in terms of color and surface texture. With the material including 5.0 parts by weight of barium silicate and 0.3 parts by weight of titanium dioxide (TiO₂) per 100 parts by weight of PPSU, it was possible to prepare pellets for injection molding.

The finished prosthesis of the present disclosure was not manufactured by CAD/CAM machining but by injection molding. In the case of CAD/CAM machining, a large portion of the raw material was discarded because a product is manufactured by cutting a block form. However, in the case of the prosthesis manufactured by injection molding as in the present disclosure, since a cutting process is not required, no waste material occurs, and post-processing is minimized. Therefore, the prosthesis can be conveniently used in the clinic and increases the satisfaction of clinicians, patients, and caregivers.

The present disclosure is also aimed at mass production of ready-made hybrid ceramic prostheses that can be rapidly implanted without having to perform post-processing during dental treatment. Therefore, an injection molding method is preferably used for the production of the hybrid ceramic prosthesis according to the present disclosure.

injection molding is a manufacturing process for making a component by injecting a molten material into a mold. It is the most commonly used method for producing plastic products. Typically, a thermoplastic resin is heated to melt, the molten resin is injected into a mold, and the mold is then cooled to product plastic products. This injection molding method automatically repeats the same processes to produce products. Therefore, preferably, the injection molding is used for mass production of ready-made products.

However, the present disclosure does not exclude the case where prostheses are manufactured by mechanical machining, i.e., a method of machining and milling a block into a predetermined shape.

Alternatively, the hybrid ceramic prosthesis as a final product, according to the present disclosure, can be manufactured by 3D-printing.

In the case where a hybrid ceramic prosthesis is manufactured by a thermosetting method using polymerization oligomers or monomers generally used in dental clinics instead of a polymer matrix, it is possible to manufacture a prosthesis having satisfiable precision through injection molding. When it is manufactured in a photocuring manner, there is an advantage of quick curing. In addition, the products can be manufactured by 3D-Printing, which has been recently extensively attracting attention.

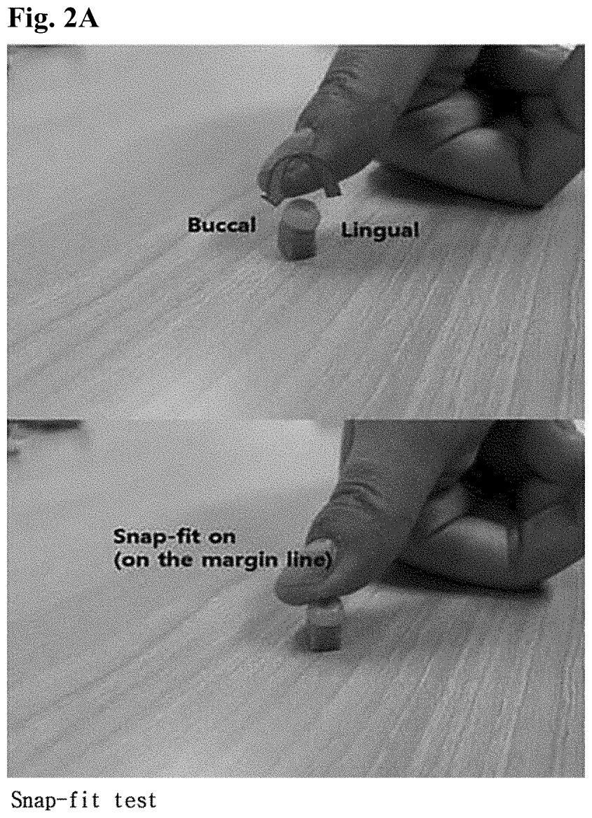

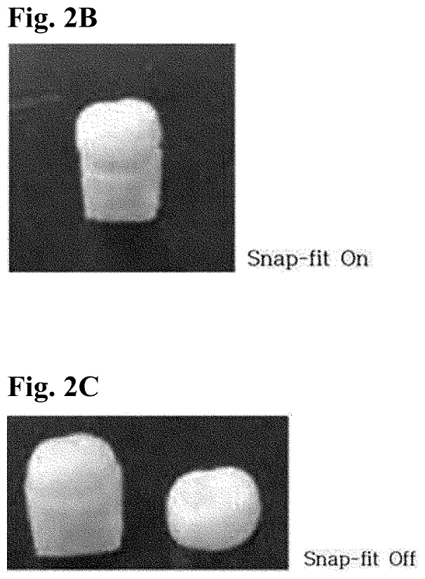

FIGS. 2A to 2C are pictures of hybrid ceramic prostheses manufactured from the pellets of FIG. 1E by injection molding.

FIG. 2B is a picture of a snap-fit on state in which the hybrid ceramic prosthesis is combined with a tooth model and FIG. 2C is a picture of a snap-fit off state in which the hybrid ceramic prosthesis is separated from the tooth model. It is confirmed that by a mechanically stable snap-fit operation, the hybrid ceramic prosthesis and the tooth model combined with sufficient bonding force.

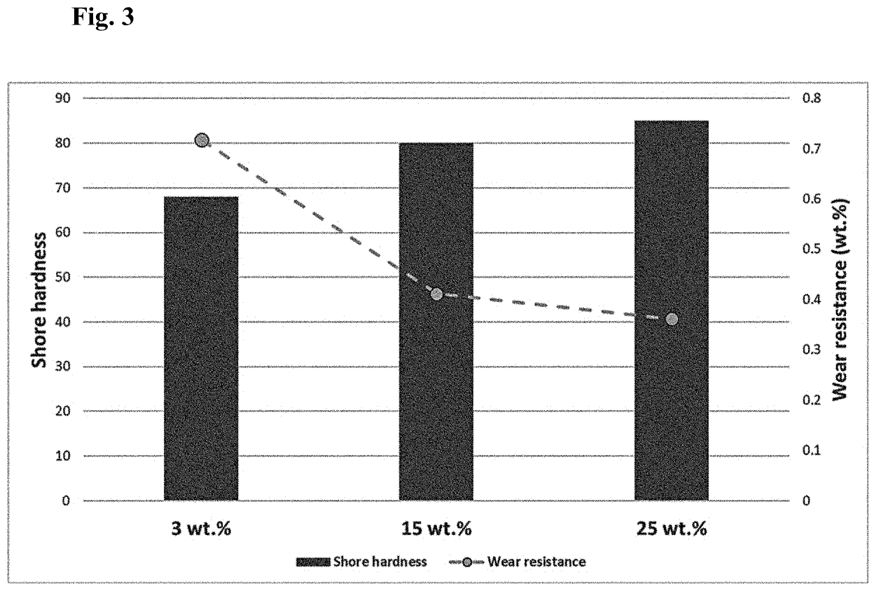

In order to evaluate the physical properties of the hybrid ceramic prosthesis of the present disclosure depending on the content of ceramic particles, the raw materials were compounded with varying contents of barium silicate ceramic particles to prepare three different compositions in which the barium silicate ceramic particle contents were 3, 15, and 25 parts by weight, respectively with respect to 100 parts by weight of PPSU. injection molding was performed using the three compositions to prepare samples, and the Shore hardness and abrasion resistance were evaluated for each sample in a manner described below. All the compositions included TiO₂ as a white pigment in an amount of 0.3 parts by weight relative to 100 parts by weight of PPSU. The obtained results are shown in FIG. 3.

(1) Shore Hardness, Durometer

Shore hardness was measured for 5 arbitrary points for each of the samples having the three compositions, and the mean value was determined as the hardness value for each sample.

(2) Wear Resistance

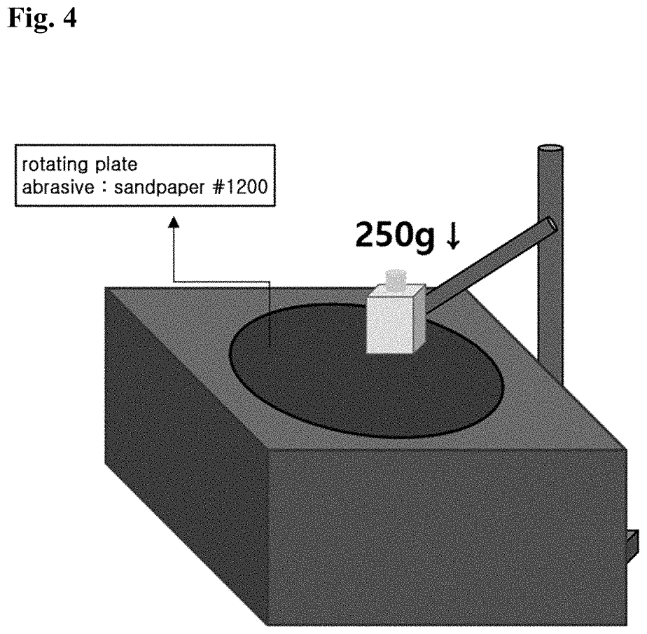

Using the apparatus illustrated in the schematic diagram of FIG. 4, #1200 SiC sandpaper as an abrasive was placed on a rotating plate, a sample (block-shaped body) was fixed so that the sample comes into contact with the abrasive, the sample was ground with a load of 250 g applied to the sample for 10 minutes while the rotating plate was rotating at a speed of 500 rpm), and the weight of the sample was measured. The wear resistance (%) was calculated from the measured weight. A total of three samples were prepared for each of the three compositions, and the wear resistance for each sample was calculated. The results of FIG. 3 are expressed as the average of the values.

As illustrated in FIG. 3, in the case of the hybrid ceramic prosthesis according to the present disclosure, as the content of ceramic particles increases, the hardness value increases and the wear resistance also increases. When the content of the ceramic particles becomes excessive, aesthetics may be impaired and snap-fit characteristics may be undesirable. Therefore, it is desirable that the content of the ceramic particles is 50 parts by weight or lower with respect to 100 parts by weight of the polymer matrix.

Specific examples and effects have been described in detail, and those who ordinarily skilled in the art will appreciate that the specific examples are only preferred embodiments and the scope of the present disclosure is not limited by the description. Thus the substantial scope of the present disclosure will be defined by the appended claims and their equivalents.

What is claimed is:

1. A hybrid ceramic prosthesis comprising a thermoplastic polymer matrix and ceramic particles dispersed in the matrix.

2. The hybrid ceramic prosthesis of claim 1, wherein the thermoplastic polymer matrix comprises one or more high performance thermoplastics selected from the group consisting of polyether ether ketone (PEEK), polyphenylsulfone (PPSU), polyamide-imide (PAI), polyphenylene sulfide (PPS), polysulfone (PSU), and polyether sulfone (PES).

3. The hybrid ceramic prosthesis of claim 1, wherein the thermoplastic polymer matrix comprises polyphenylsulfone (PPSU).

4. The hybrid ceramic prosthesis of claim 3, wherein the polyphenylsulfone (PPSU) satisfies conditions, based on a film thickness of 2 mm or less: a light transmittance of at least 75% and a haze of at most 5.1%, and a yellowness index of 28 or less.

5. The hybrid ceramic prosthesis of claim 4, wherein the PPSU satisfies conditions, based on a film thickness of 2 mm or less: a tensile strength of 68 to 72 MPa, a tensile modulus of 2.0 to 2.4 GPa, a tensile elongation at break of 60% to 120%, a flexural strength of 88 to 95 MPa, and an impact resistance (Izod impact, notched) of 680 to 700 J/m.

6. The hybrid ceramic prosthesis of claim 1, wherein the ceramic particles are included in an amount of 3 to 50 parts by weight relative to 100 parts by weight of the thermoplastic polymer matrix.

7. The hybrid ceramic prosthesis of claim 1, wherein the ceramic particles comprise silicate-based glass ceramic particles.

8. The hybrid ceramic prosthesis of claim 7, wherein the silicate-based glass ceramic particles are lithium disilicate glass ceramic particles.

9. The hybrid ceramic prosthesis of claim 7, wherein the silicate-based glass ceramic particles are barium silicate glass ceramic particles.

10. The hybrid ceramic prosthesis of claim 1, further comprising a white pigment.

11. The hybrid ceramic prosthesis of claim 10, wherein the white pigment comprises one or more selected from the group consisting of titanium dioxide (TiO₂), zinc oxide (ZnO), and zinc phosphate (Zn ₃(PO₄) 2).

12. The hybrid ceramic prosthesis of claim 10, wherein the white pigment comprises titanium dioxide (TiO₂).

13. The hybrid ceramic prosthesis of claim 12, wherein the white pigment is included an amount of 0.05 to 1.0 part by weight relative to 100 parts by weight of the thermoplastic polymer matrix.

14. The hybrid ceramic prosthesis of claim 1, the hybrid ceramic prosthesis is an injection molded product comprising 3.0 to 50.0 parts by weight of barium silicate and 0.05 to 1.0 part by weight of titanium dioxide (TiO₂), per 100 parts by weight of polyphenylsulfone (PPSU).

15. The hybrid ceramic prosthesis of claim 1, wherein the hybrid ceramic prosthesis is a primary crown.

16. The hybrid ceramic prosthesis of claim 1, wherein the prosthesis is coupled with a prepped tooth by a snap-fit fastening method.
